SEQUENCE LISTING (64 sequences)

SEQ ID NO: 1 - DNA, 22 bp, Artificial Sequence
  Description of Artificial Sequence: Synthetic oligonucleotide
ttcttcaggatcttattcagca

SEQ ID NO: 2 - DNA, 22 bp, Artificial Sequence
  Description of Artificial Sequence: Synthetic oligonucleotide
attcttcaggatcttattcagc

SEQ ID NO: 3 - DNA, 21 bp, Artificial Sequence
  Description of Artificial Sequence: Synthetic oligonucleotide
tcttcaggatcttattcagca

SEQ ID NO: 4 - DNA, 21 bp, Artificial Sequence
  Description of Artificial Sequence: Synthetic oligonucleotide
ttcttcaggatcttattcagc

SEQ ID NO: 5 - DNA, 20 bp, Artificial Sequence
  Description of Artificial Sequence: Synthetic oligonucleotide
cttcaggatcttattcagca

SEQ ID NO: 6 - DNA, 20 bp, Artificial Sequence
  Description of Artificial Sequence: Synthetic oligonucleotide
ttcttcaggatcttattcag

SEQ ID NO: 7 - DNA, 19 bp, Artificial Sequence
  Description of Artificial Sequence: Synthetic oligonucleotide
cttcaggatcttattcagc

SEQ ID NO: 8 - DNA, 19 bp, Artificial Sequence
  Description of Artificial Sequence: Synthetic oligonucleotide
tcttcaggatcttattcag

SEQ ID NO: 9 - DNA, 18 bp, Artificial Sequence
  Description of Artificial Sequence: Synthetic oligonucleotide
ttcaggatcttattcagc

SEQ ID NO: 10 - DNA, 18 bp, Artificial Sequence
  Description of Artificial Sequence: Synthetic oligonucleotide
cttcaggatcttattcag

SEQ ID NO: 11 - DNA, 18 bp, Artificial Sequence
  Description of Artificial Sequence: Synthetic oligonucleotide
tcttcaggatcttattca

SEQ ID NO: 12 - DNA, 17 bp, Artificial Sequence
  Description of Artificial Sequence: Synthetic oligonucleotide
tcaggatcttattcagc

SEQ ID NO: 13 - DNA, 17 bp, Artificial Sequence
  Description of Artificial Sequence: Synthetic oligonucleotide
tcttcaggatcttattc

SEQ ID NO: 14 - DNA, 20 bp, Artificial Sequence
  Description of Artificial Sequence: Synthetic oligonucleotide
tcttcaggatcttattcagc

SEQ ID NO: 15 - DNA, 22 bp, Artificial Sequence
  Description of Artificial Sequence: Synthetic oligonucleotide
aatataagcatcacaaagtacc

SEQ ID NO: 16 - DNA, 21 bp, Artificial Sequence
  Description of Artificial Sequence: Synthetic oligonucleotide
ataagcatcacaaagtacctc

SEQ ID NO: 17 - DNA, 21 bp, Artificial Sequence
  Description of Artificial Sequence: Synthetic oligonucleotide
tataagcatcacaaagtacct

SEQ ID NO: 18 - DNA, 21 bp, Artificial Sequence
  Description of Artificial Sequence: Synthetic oligonucleotide
atataagcatcacaaagtacc

SEQ ID NO: 19 - DNA, 21 bp, Artificial Sequence
  Description of Artificial Sequence: Synthetic oligonucleotide
aatataagcatcacaaagtac

SEQ ID NO: 20 - DNA, 20 bp, Artificial Sequence
  Description of Artificial Sequence: Synthetic oligonucleotide
taagcatcacaaagtacctc

SEQ ID NO: 21 - DNA, 20 bp, Artificial Sequence
  Description of Artificial Sequence: Synthetic oligonucleotide
ataagcatcacaaagtacct

SEQ ID NO: 22 - DNA, 20 bp, Artificial Sequence
  Description of Artificial Sequence: Synthetic oligonucleotide
tataagcatcacaaagtacc

SEQ ID NO: 23 - DNA, 20 bp, Artificial Sequence
  Description of Artificial Sequence: Synthetic oligonucleotide
atataagcatcacaaagtac

SEQ ID NO: 24 - DNA, 19 bp, Artificial Sequence
  Description of Artificial Sequence: Synthetic oligonucleotide
aagcatcacaaagtacctc

SEQ ID NO: 25 - DNA, 19 bp, Artificial Sequence
  Description of Artificial Sequence: Synthetic oligonucleotide
taagcatcacaaagtacct

SEQ ID NO: 26 - DNA, 19 bp, Artificial Sequence
  Description of Artificial Sequence: Synthetic oligonucleotide
ataagcatcacaaagtacc

SEQ ID NO: 27 - DNA, 19 bp, Artificial Sequence
  Description of Artificial Sequence: Synthetic oligonucleotide
tataagcatcacaaagtac

SEQ ID NO: 28 - DNA, 19 bp, Artificial Sequence
  Description of Artificial Sequence: Synthetic oligonucleotide
atataagcatcacaaagta

SEQ ID NO: 29 - DNA, 18 bp, Artificial Sequence
  Description of Artificial Sequence: Synthetic oligonucleotide
agcatcacaaagtacctc

SEQ ID NO: 30 - DNA, 18 bp, Artificial Sequence
  Description of Artificial Sequence: Synthetic oligonucleotide
aagcatcacaaagtacct

SEQ ID NO: 31 - DNA, 18 bp, Artificial Sequence
  Description of Artificial Sequence: Synthetic oligonucleotide
taagcatcacaaagtacc

SEQ ID NO: 32 - DNA, 18 bp, Artificial Sequence
  Description of Artificial Sequence: Synthetic oligonucleotide
ataagcatcacaaagtac

SEQ ID NO: 33 - DNA, 18 bp, Artificial Sequence
  Description of Artificial Sequence: Synthetic oligonucleotide
tataagcatcacaaagta

SEQ ID NO: 34 - DNA, 17 bp, Artificial Sequence
  Description of Artificial Sequence: Synthetic oligonucleotide
aagcatcacaaagtacc

SEQ ID NO: 35 - DNA, 22 bp, Artificial Sequence
  Description of Artificial Sequence: Synthetic oligonucleotide
atataagcatcacaaagtacct

SEQ ID NO: 36 - DNA, 21 bp, Artificial Sequence
  Description of Artificial Sequence: Synthetic oligonucleotide
gtctgaaatgaatgactcaaa

SEQ ID NO: 37 - DNA, 21 bp, Artificial Sequence
  Description of Artificial Sequence: Synthetic oligonucleotide
ggaatattcttcaggatctta

SEQ ID NO: 38 - DNA, 20 bp, Artificial Sequence
  Description of Artificial Sequence: Synthetic oligonucleotide
ctccaggaatattcttcagg

SEQ ID NO: 39 - DNA, 20 bp, Artificial Sequence
  Description of Artificial Sequence: Synthetic oligonucleotide
gcatgtccttacccgttttt

SEQ ID NO: 40 - DNA, 20 bp, Artificial Sequence
  Description of Artificial Sequence: Synthetic oligonucleotide
aatgcatgtccttacccgtt

SEQ ID NO: 41 - DNA, 22 bp, Artificial Sequence
  Description of Artificial Sequence: Synthetic oligonucleotide
acttgaatgcatgtccttaccc

SEQ ID NO: 42 - DNA, 20 bp, Artificial Sequence
  Description of Artificial Sequence: Synthetic oligonucleotide
ttgaatgcatgtccttaccc

SEQ ID NO: 43 - DNA, 18 bp, Artificial Sequence
  Description of Artificial Sequence: Synthetic oligonucleotide
gaatgcatgtccttaccc

SEQ ID NO: 44 - DNA, 16 bp, Artificial Sequence
  Description of Artificial Sequence: Synthetic oligonucleotide
atgcatgtccttaccc

SEQ ID NO: 45 - DNA, 14 bp, Artificial Sequence
  Description of Artificial Sequence: Synthetic oligonucleotide
gcatgtccttaccc

SEQ ID NO: 46 - DNA, 20 bp, Artificial Sequence
  Description of Artificial Sequence: Synthetic oligonucleotide
acttgaatgcatgtccttac

SEQ ID NO: 47 - DNA, 20 bp, Artificial Sequence
  Description of Artificial Sequence: Synthetic oligonucleotide
ggacttgaatgcatgtcctt

SEQ ID NO: 48 - DNA, 22 bp, Artificial Sequence
  Description of Artificial Sequence: Synthetic oligonucleotide
tcttcaggatcttattcagcat

SEQ ID NO: 49 - DNA, 23 bp, Artificial Sequence
  Description of Artificial Sequence: Synthetic oligonucleotide
tacctcaacacttctgaccatct

SEQ ID NO: 50 - DNA, 22 bp, Artificial Sequence
  Description of Artificial Sequence: Synthetic oligonucleotide
agtacctcaacacttctgacca

SEQ ID NO: 51 - DNA, 22 bp, Artificial Sequence
  Description of Artificial Sequence: Synthetic oligonucleotide
caaagtacctcaacacttctga

SEQ ID NO: 52 - DNA, 22 bp, Artificial Sequence
  Description of Artificial Sequence: Synthetic oligonucleotide
tcacaaagtacctcaacacttc

SEQ ID NO: 53 - DNA, 22 bp, Artificial Sequence
  Description of Artificial Sequence: Synthetic oligonucleotide
gcatcacaaagtacctcaacac

SEQ ID NO: 54 - DNA, 25 bp, Artificial Sequence
  Description of Artificial Sequence: Synthetic oligonucleotide
taagcatcacaaagtacctcaacac

SEQ ID NO: 55 - DNA, 22 bp, Artificial Sequence
  Description of Artificial Sequence: Synthetic oligonucleotide
taagcatcacaaagtacctcaa

SEQ ID NO: 56 - DNA, 22 bp, Artificial Sequence
  Description of Artificial Sequence: Synthetic oligonucleotide
ataatataagcatcacaaagta

SEQ ID NO: 57 - DNA, 22 bp, Artificial Sequence
  Description of Artificial Sequence: Synthetic oligonucleotide
tgctaatataatataagcatca

SEQ ID NO: 58 - DNA, 22 bp, Artificial Sequence
  Description of Artificial Sequence: Synthetic oligonucleotide
ctaagtttgctaatataatata

SEQ ID NO: 59 - DNA, 22 bp, Artificial Sequence
  Description of Artificial Sequence: Synthetic oligonucleotide
tctctacagagagtaacacagc

SEQ ID NO: 60 - DNA, 129 bp, Homo sapiens
aggaggcctatcagaagctttaatgtagtggagagcatttgttttcttggtgttgggaggcagttttacctttgagtcattcatttcagacttaccattggggtagaatggtgttgacattagcaggag

SEQ ID NO: 61 - DNA, 129 bp, Homo sapiens
ttggagaagataaaaattcttcatattccattttaatgctgaataagatcctgaagaatattcctggagaaagtatgaatgggatatagaaaaacgggtaaggacatgcattcaagtccaagcttgtgc

SEQ ID NO: 62 - DNA, 153 bp, Homo sapiens
tttcagacttaccattggggtagaatggtgttgacattagcaggagttggagaagataaaaattcttcatattccattttaatgctgaataagatcctgaagaatattcctggagaaagtatgaatgggatatagaaaaacgggtaaggacat

SEQ ID NO: 63 - DNA, 101 bp, Homo sapiens
gtaggagacctcagatggtcagaagtgttgaggcactttgtgatgcttatattatattagcaaacttagatgccactcagtggaagactcagagaagtatg

SEQ ID NO: 64 - protein, 33 aa, Homo sapiens
SRRPQMVRSVEALCDAYIILANLDATQWKTQRK

(19) United States
(12) Patent Application Publication
(10) Pub. No.: US 2023/0174979 A1
(43) Pub. Date: Jun. 8, 2023

(54) METHODS AND COMPOSITIONS FOR TREATING ATAXIA TELANGIECTASIA

(71) Applicant: Children’s Medical Center Corporation, Boston, MA (US)

(72) Inventors: Timothy W. YU, Boston, MA (US); Jinkuk KIM, Boston, MA (US)

(73) Assignee: Children’s Medical Center Corporation, Boston, MA (US)

(21) Appl. No.: 17/767,340

(22) PCT Filed: Oct. 8, 2020
(86) PCT No.: PCT/US2020/054718

§ 371 Date: Apr. 7, 2022

Related U.S. Application Data
(60) Provisional application No. 62/912,997, filed on Oct. 9, 2019.

(51) Int. Cl. C12N 15/113; A61P 25/00

(52) U.S. Cl. CPC C12N 15/113; A61P 25/00; C12N 2310/11; C12N 2310/315; C12N 2310/3341; C12N 2320/33

(57) ABSTRACT

Provided herein are antisense oligonucleotides and prophylactic and therapeutic methods featuring such oligonucleotides. These oligonucleotides and methods are useful for treating or preventing ataxia telangiectasia in a subject. Specifically, the disclosure provides antisense nucleobase oligomers each comprising (8-40) nucleobases, wherein at least 90% of said nucleobases or more than (8) consecutive nucleobases of the oligomer are complementary to a nucleic acid sequence in an Ataxia-Telangiectasia Mutated (ATM) allele comprising a mutation associated with aberrant splicing.

### CROSS-REFERENCE TO RELATED APPLICATIONS

This application is the U.S. National Stage Application, pursuant to 35 U.S.C. §371 of PCT International Application No. PCT/US2020/054718, filed Oct. 8, 2020 designating the United States and published in English, which claims the benefit of and priority to U.S. Provisional Application No.: 62/912,997, filed Oct. 9, 2019, the entire contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 19, 2020, is named 167705_020301_PCT_SL.txt and is 15,644 bytes in size.

### BACKGROUND OF THE INVENTION

Ataxia Telangiectasia (A-T) is an incurable autosomal recessive disease caused by mutations in the Ataxia-Telangiectasia Mutated (ATM) gene. Ataxia refers to poor coordination and telangiectasia to small dilated blood vessels, both of which are hallmarks of the disease. A-T is characterized by neurodegeneration in the central nervous system, immune deficiency, and skin and ocular telangiectasias. By age two, an A-T patient can display delayed walking or wobbly gait, problems standing or sitting still, and truncal sway. As childhood progresses, the patient may suffer from involuntary movements, worsening gait, uncoordinated eye movement, fine motor difficulty, and dysarthria. Typically, a wheelchair is required by age 10 and life expectancy is only approximately 25 years. Methods of treating A-T are urgently required.

### SUMMARY OF THE INVENTION

As described below, the present invention features antisense nucleobase oligomers (e.g., oligonucleotides) that restore wild-type splicing of an ATM gene comprising a mutation associated with Ataxia Telangiectasia, and methods of using such oligomers in treating A-T.

One aspect of the present invention provides an antisense nucleobase oligomer that includes 8-40 nucleobases, wherein at least 90% of the nucleobases or more than 8 consecutive nucleobases of the oligomer are complementary to a nucleic acid sequence in an Ataxia-Telangiectasia Mutated (ATM) allele having a mutation associated with aberrant splicing.

In an embodiment, the antisense nucleobase oligomer includes a modified linkage selected from the group consisting of phosphorothioate, methylphosphonate, phosphodiester, phosphotriester, and phosphorodithioate linkages. In an embodiment, the modified linkage is a phosphorothioate linkage. In some embodiments, the antisense nucleobase oligomer is a mixture of one or more stereopure oligomers with a defined sequence. In some embodiments, the antisense nucleobase oligomer includes a modified nucleobase. In some embodiments, the modified nucleobase is 5-methyl cytosine. In some embodiments, the antisense nucleobase oligomer also includes at least one modified sugar moiety. In some embodiments, the modified sugar moiety is a 2′-O-methoxyethyl group, a 2′-O-methyl, 2′-dimethylaminooxyethoxy, 2′-dimethylaminoethoxyethoxy, 2′-fluoro or 2′-acetamide modification group. In some embodiments, the oligomer includes locked nucleic acids. In some embodiments, the oligomer is a morpholino, thiomorpholino, or peptide nucleic acid. In some embodiments, the mutation is a NM_000051.3(ATM):c0.5762ins137 mutation or a NM_000051.3(ATM):c0.7865C>T mutation. In some embodiments, the antisense nucleobase oligomer comprises or consists of a nucleic acid sequence having at least 80% sequence identify to a nucleotide sequence selected from the group listed in Table 1 or Table 2. In some embodiments, the antisense nucleobase oligomer comprises or consists of a nucleic acid sequence at least 90% sequence identity to a nucleotide sequence selected from the group listed in Table 1 or Table 2. In some embodiments, the antisense nucleobase oligomer comprises or consists of a nucleic acid sequence having complete sequence identity to a nucleotide sequence selected from the group listed in Table 1 or Table 2. In some embodiments, the antisense nucleobase oligomer comprises at least 10 nucleobases of the following sequences or consists of the following sequences: TTCTTCAGGATCTTATTCAGCA (SEQ ID NO: 1); ATTCTTCAGGATCTTATTCAGC (SEQ ID NO: 2); TCTTCAGGATCTTATTCAGCA (SEQ ID NO: 3); TTCTTCAGGATCTTATTCAGC (SEQ ID NO: 4); CTTCAGGATCTTATTCAGCA (SEQ ID NO: 5); TTCTTCAGGATCTTATTCAG (SEQ ID NO: 6); CTTCAGGATCTTATTCAGC (SEQ ID NO: 7); TCTTCAGGATCTTATTCAG (SEQ ID NO: 8); TTCAGGATCTTATTCAGC (SEQ ID NO: 9); CTTCAGGATCTTATTCAG (SEQ ID NO: 10); TCTTCAGGATCTTATTCA (SEQ ID NO: 11); TCAGGATCTTATTCAGC (SEQ ID NO: 12); and TCTTCAGGATCTTATTC (SEQ ID NO: 13). In some embodiments, the antisense nucleobase oligomer comprises or consists of TCTTCAGGATCTTATTCAGC (SEQ ID NO: 14). In some embodiments, the antisense nucleobase oligomer comprises at least 10 nucleobases of the following sequences or consists of the following sequences: AATATAAGCATCACAAAGTACC (SEQ ID NO: 15); ATAAGCATCACAAAGTACCTC (SEQ ID NO: 16); TATAAGCATCACAAAGTACCT (SEQ ID NO: 17); ATATAAGCATCACAAAGTACC (SEQ ID NO: 18); AATATAAGCATCACAAAGTAC (SEQ ID NO: 19); TAAGCATCACAAAGTACCTC (SEQ ID NO: 20); ATAAGCATCACAAAGTACCT (SEQ ID NO: 21); TATAAGCATCACAAAGTACC (SEQ ID NO: 22); ATATAAGCATCACAAAGTAC (SEQ ID NO: 23); AAGCATCACAAAGTACCTC (SEQ ID NO: 24); TAAGCATCACAAAGTACCT (SEQ ID NO: 25); ATAAGCATCACAAAGTACC (SEQ ID NO: 26); TATAAGCATCACAAAGTAC (SEQ ID NO: 27); ATATAAGCATCACAAAGTA (SEQ ID NO: 28); AGCATCACAAAGTACCTC (SEQ ID NO: 29); AAGCATCACAAAGTACCT (SEQ ID NO: 30); TAAGCATCACAAAGTACC (SEQ ID NO: 31); ATAAGCATCACAAAGTAC (SEQ ID NO: 32); TATAAGCATCACAAAGTA (SEQ ID NO: 33); and AAGCATCACAAAGTACC (SEQ ID NO: 34). In some embodiments, the antisense nucleobase oligomer comprises or consists of ATATAAGCATCACAAAGTACCT (SEQ ID NO: 35) or AATATAAGCATCACAAAGTAC (SEQ ID NO: 19). Some embodiments provide a set of antisense nucleobase oligomers that include two or more of the antisense nucleobase oligomers described above. Another embodiment provides a pharmaceutical composition that includes an effective amount of any of the antisense nucleobase oligomers described above and a pharmaceutically acceptable excipient.

Another aspect of the present invention provides a method of restoring wild-type splicing of an ATM allele having a mutation associated with ataxia telangiectasia in a cell, and the method includes contacting the cell with an effective amount any of the antisense nucleobase oligomer described above, thereby restoring wild-type splicing. In some embodiments of the method, the cell is heterozygous or homozygous for the mutation. In some embodiments, the mutation is in a splice acceptor site, a splice donor site, or a splicing regulatory element. In some embodiments, the splicing regulatory element is an exonic splicing enhancer (ESE).

Another aspect of the present invention provides a method of treating ataxia telangiectasia in a subject, and the method includes administering to the subject an effective amount of any of the antisense nucleobase oligomers described above. In some embodiments, the subject comprises a mutation associated with ataxia telangiectasia. In some embodiments, the subject is heterozygous or homozygous for the mutation. In some embodiments of the methods presented herein , the mutation is a NM_000051.3(ATM):c0.5762ins137 mutation. In some embodiments, the mutation is a NM_000051.3(ATM):c0.7865C>T mutation.

Some aspects of the present invention provide an isolated cell comprising any of the antisense oligomer described above and a mutation associated with ataxia telangiectasia. In some embodiments, the cell comprises a NM_000051.3(ATM):c0.5762ins137 or NM_000051.3(ATM):c0.7865C>T mutation. In some embodiments, the cell is a fibroblast. In some embodiments, the cell is derived from a subject having or suspected of having ataxia telangiectasia.

Another aspect provides a kit comprising any of the antisense nucleobase oligomers described above and directions for administering the antisense nucleobase oligomer to a subject.

Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In one embodiment, the agent is an inhibitory nucleic acid molecule, such as an antisense oligonucleotide.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, or a 50% or greater change in expression levels.

By “ameliorate” is meant decrease, suppress, attenuate, diminish, arrest, or stabilize the development or progression of a disease. In one embodiment, the disease is ataxia telangiectasia.

By “analog” is meant a molecule that is not identical, but has analogous functional or structural features. For example, a polypeptide analog retains the biological activity of a corresponding naturally-occurring polypeptide, while having certain biochemical modifications that enhance the analog’s function relative to a naturally occurring polypeptide. Such biochemical modifications could increase the analog’s protease resistance, membrane permeability, or half-life, without altering, for example, ligand binding. An analog may include an unnatural amino acid.

“Antisense nucleobase oligomer” is meant a molecule that includes a chain of at least eight nucleobases joined together by linkage groups, wherein at least a portion of the oligomer is complementary to a target sequence. Included in this definition are natural and non-natural oligonucleotides, both modified and unmodified, as well as oligonucleotide mimetics such as Protein Nucleic Acids and locked nucleic acids. Numerous nucleobases and linkage groups may be employed in the nucleobase oligomers of the invention, including those described in detail herein. In an embodiment, the nucleobase oligomer is an antisense oligonucleotide that may contain modified bases, a modified backbone, or any other modification described herein or known in the art. Table 1 provides a list of antisense oligonucleotides directed against pseudo-exon inclusion in a mature Ataxia Telangiectasia Mutated (ATM) kinase transcript arising from a mutation in ATM (i.e., NM_000051.3(ATM):c0.5762_5763insNG_009830.1:g0.91138_91274 (alternate: NM_000051.3(ATM):c0.5762ins137), a mutation that is found in 18% of patients with A-T in the United Kingdom. Table 2 provides a list of antisense oligonucleotides (also referred to as “ASOs”) directed against ATM alleles having the NM_000051.3(ATM):c0.7865C>T mutation that results in truncation of exon 53.

TABLE 1

Antisense Oligonucleotides Targeting NM_000051.3(ATM):c0.5762ins137 Mutation

| ID | Sequence (5′ to 3′) | SEQ ID NO |
|---|---|---|
| AT042 | GTCTGAAATGAATGACTCAAA | 36 |
| AT043 | TCTTCAGGATCTTATTCAGC | 14 |
| AT044 | GGAATATTCTTCAGGATCTTA | 37 |
| AT045 | CTCCAGGAATATTCTTCAGG | 38 |
| AT046 | GCATGTCCTTACCCGTTTTT | 39 |
| AT047 | AATGCATGTCCTTACCCGTT | 40 |
| AT048 | ACTTGAATGCATGTCCTTACCC | 41 |
| AT049 | TTGAATGCATGTCCTTACCC | 42 |
| AT050 | GAATGCATGTCCTTACCC | 43 |
| AT051 | ATGCATGTCCTTACCC | 44 |
| AT052 | GCATGTCCTTACCC | 45 |
| AT053 | ACTTGAATGCATGTCCTTAC | 46 |
| AT054 | GGACTTGAATGCATGTCCTT | 47 |
| AT055 | TCTTCAGGATCTTATTCAGCAT | 48 |
| AT056 | TTCTTCAGGATCTTATTCAGCA | 1 |
| AT057 | ATTCTTCAGGATCTTATTCAGC | 2 |
| AT058 | TCTTCAGGATCTTATTCAGCA | 3 |
| AT059 | TTCTTCAGGATCTTATTCAGC | 4 |
| AT060 | CTTCAGGATCTTATTCAGCA | 5 |
| AT061 | TTCTTCAGGATCTTATTCAG | 6 |
| AT062 | CTTCAGGATCTTATTCAGC | 7 |
| AT063 | TCTTCAGGATCTTATTCAG | 8 |
| AT064 | TTCAGGATCTTATTCAGC | 9 |
| AT065 | CTTCAGGATCTTATTCAG | 10 |
| AT066 | TCTTCAGGATCTTATTCA | 11 |
| AT067 | TCAGGATCTTATTCAGC | 12 |
| AT068 | TCTTCAGGATCTTATTC | 13 |

TABLE 2

Antisense Oligonucleotides Targeting NM_000051.3(ATM):c0.7865C>T Mutation

| ID | Sequence (5′ to 3′) | SEQ ID NO |
|---|---|---|
| AT001 | TACCTCAACACTTCTGACCATCT | 49 |
| AT002 | AGTACCTCAACACTTCTGACCA | 50 |
| AT003 | CAAAGTACCTCAACACTTCTGA | 51 |
| AT004 | TCACAAAGTACCTCAACACTTC | 52 |
| AT005 | GCATCACAAAGTACCTCAACAC | 53 |
| AT006 | TAAGCATCACAAAGTACCTCAACAC | 54 |
| AT007 | TAAGCATCACAAAGTACCTCAA | 55 |
| AT008 | ATATAAGCATCACAAAGTACCT | 35 |
| AT009 | ATAATATAAGCATCACAAAGTA | 56 |
| AT010 | TGCTAATATAATATAAGCATCA | 57 |
| AT011 | CTAAGTTTGCTAATATAATATA | 58 |
| AT012 | TCTCTACAGAGAGTAACACAGC | 59 |
| AT022 | AATATAAGCATCACAAAGTACC | 15 |
| AT023 | ATAAGCATCACAAAGTACCTC | 16 |
| AT024 | TATAAGCATCACAAAGTACCT | 17 |
| AT025 | ATATAAGCATCACAAAGTACC | 18 |
| AT026 | AATATAAGCATCACAAAGTAC | 19 |
| AT027 | TAAGCATCACAAAGTACCTC | 20 |
| AT028 | ATAAGCATCACAAAGTACCT | 21 |
| AT029 | TATAAGCATCACAAAGTACC | 22 |
| AT030 | ATATAAGCATCACAAAGTAC | 23 |
| AT031 | AAGCATCACAAAGTACCTC | 24 |
| AT032 | TAAGCATCACAAAGTACCT | 25 |
| AT033 | ATAAGCATCACAAAGTACC | 26 |
| AT034 | TATAAGCATCACAAAGTAC | 27 |
| AT035 | ATATAAGCATCACAAAGTA | 28 |
| AT036 | AGCATCACAAAGTACCTC | 29 |
| AT037 | AAGCATCACAAAGTACCT | 30 |
| AT038 | TAAGCATCACAAAGTACC | 31 |
| AT039 | ATAAGCATCACAAAGTAC | 32 |
| AT040 | TATAAGCATCACAAAGTA | 33 |
| AT041 | AAGCATCACAAAGTACC | 34 |

In some embodiments, the oligonucleotide comprises a 2′-O-methoxyethyl (2′MOE) modification. In some embodiments, the oligonucleotide comprises a gene. In some embodiments, the oligonucleotide comprises a 5-methyluridine. In some embodiments, the oligonucleotide comprises a phosphorothioate linkage between the nucleobases. In some embodiments, the oligonucleotide comprises a phosphorothioate backbone. Phosphorothioate linkages have two stereoisomers (phosphodiester linkages are prochiral). Antisense oligonucleotides (ASOs) that are synthesized by standard methods will comprise diastereomers. The stereoisomers that make up the diastereomers can be isolated, yielding two stereopure oligomer populations. “Stereopure” antisense oligonucleotides comprise a single stereoisomer. In some embodiments, the antisense nucleobase oligomer is a mixture of one or more stereopure molecules with a defined sequence.

In this disclosure, “comprises,” “comprising,” “containing,” and “having” and the like can have the meaning ascribed to them in U.S. Patent law and can mean “includes,” “including,” and the like; “consisting essentially of” or “consists essentially” likewise has the meaning ascribed in U.S. Patent law, and the term is open-ended, allowing for the presence of more than that which is recited so long as basic or novel characteristics of that which is recited is not changed by the presence of more than that which is recited, but excludes prior art embodiments.

“Detect” refers to identifying the presence, absence, or amount of the analyte to be detected. In one embodiment, a mutation in an ATM polypeptide or polynucleotide is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In one embodiment, a disease is ataxia telangiectasia (A-T), a recessive disease associated with mutations in the ATM gene.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount of an antisense oligonucleotide is the amount required to block abnormal splicing in a cell expressing an ATM polynucleotide comprising a mutation.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid molecule” is meant a double-stranded or single-stranded oligonucleotide (e.g., siRNA, shRNA, or antisense RNA), or a fragment thereof that when administered to a mammalian cell results in a decrease (e.g., by 10%, 25%, 50%, 75%, or even 90-100%) in the expression of a target gene. Typically, an inhibitory nucleic acid comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein. In one embodiment, an antisense oligonucleotide comprises at least about 5, 10, 11, 12, 13, 14, 15, 16, 17, 18, 19, 20, 21, 22, 23, 24, 25, 26, 27, 28, 29, 30, 35 or more nucleobases complementary to a target sequence. In one embodiment, the oligonucleotide comprises 18-22 nucleobases.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule (e.g., DNA, RNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level, activity, or structure that is associated with a disease or disorder.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition. In one embodiment, a reference refers to the expression of an ATM polypeptide or polynucleotide in an untreated control cell expressing an ATM polypeptide or polynucleotide comprising a mutation associated with A-T. In another embodiment, a reference refers to the expression of an ATM polypeptide or polynucleotide in a cell expressing a wild-type ATM polypeptide or polynucleotide.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 10, 15, or 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 5, 10, 15, 20, or 50 nucleotides, at least about 60 nucleotides, at least about 75 nucleotides, at least about 100 nucleotides, or at least about 300 nucleotides or any integer thereabout or therebetween.

By “specifically binds” is meant a polynucleotide that recognizes and binds a complementary sequence of the invention. An antisense oligonucleotide may specifically bind its perfect complement or a sequence to which it is not perfectly complementary. In one embodiment, the antisense oligonucleotide specifically binds a polynucleotide that comprises 1, 2, 3, 4, 5 or more bases that are not perfectly complementary to the anti-sense oligonucleotide.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an: embodiment, hybridization will occur at 30° C. in 750 mM NaCl, 75 mM trisodium citrate, and 1% SDS. In an embodiment, hybridization will occur at 37° C. in 500 mM NaCl, 50 mM trisodium citrate, 1% SDS, 35% formamide, and 100 µg/ml denatured salmon sperm DNA (ssDNA). In another embodiment, hybridization will occur at 42° C. in 250 mM NaCl, 25 mM trisodium citrate, 1% SDS, 50% formamide, and 200 µg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, in some embodiments, stringent salt concentration for the wash steps will be less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80%, 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disease and/or symptoms associated therewith. It will be appreciated, although not precluded, that treating a disease, disorder, or condition does not require that the disease, disorder, condition, or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B illustrate the location and splicing impact of the NM_000051.3(ATM):c0.5762ins137 mutation. FIG. 1A is a diagram of the region of chromosome 11 that comprises the A to G mutation. FIG. 1B is an illustration of the aberrant splicing caused by the NM_000051.3(ATM):c0.5762ins137 mutation. “X” denotes the location of the mutation. “MT” denotes mutant. “WT” denotes wild-type.

FIG. 2 is a diagram that illustrates the location and splicing impact of the NM_000051.3(ATM):c.7865C>T mutation. The mutation creates a novel splice site that truncates 64 nucleotides from the 3′ end of exon 53.

FIGS. 3A and 3B illustrate the therapeutic approach to restoring wild-type splicing in cells that have an ATM allele with the NM_000051.3(ATM):c0.5762ins137 mutation. FIG. 3A is a schematic of the therapeutic approach. “ASO” denotes antisense oligonucleotide. AT042-AT054 are identifiers for individual antisense oligonucleotides. “X” denotes the site of the mutation. “STOP” denotes a premature stop codon in the pseudo exon. FIG. 3B is a diagram of regions in the ATM gene that have a nucleic acid sequence complementary to the antisense oligonucleotides, AT042-AT054. “ESE” denotes exonic splicing enhancer. “ESS” denotes exonic splicing silencer. Figure discloses SEQ ID NOS 60-61, respectively, in order of appearance.

FIGS. 4A and 4B illustrate that wild-type splicing is restored in cultured fibroblast cells from an A-T patient with a NM_000051.3(ATM):c0.5762ins137 mutation (heterozygous) transfected with antisense oligonucleotides AT042-AT054. FIG. 4A is a gel image of RT-PCR assay targeting the ATM gene. “772” and “774” are negative control scrambled antisense oligonucleotides. “E38” denotes exon 38, and “E39” denotes exon 39. The black rectangle highlights the normal splicing achieved using AT043. FIG. 4B is a graph that quantifies the percentage of wild-type splicing shown in FIG. 5A.

FIGS. 5A and 5B depict antisense oligonucleotides AT043 and AT055-AT068 and their effectiveness in restoring wild-type splicing to cells having a NM_000051.3(ATM):c0.5762ins137 mutation in the ATM gene. FIG. 5A is a diagram showing where candidate antisense oligonucleotides AT055-AT068 (derivatives of AT043) map to the ATM gene and in relation to antisense oligonucleotide AT043. Figure discloses SEQ ID NO: 62. “ESS” denotes exonic splicing silencer. “ESE” denotes exonic splicing enhancer. “Cryptic exon” refers to the pseudo-exon between exons 38 and 39. FIG. 5B is a graph depicting the percentage of wild-type splicing observed after treating fibroblast cells having the NM_000051.3(ATM):c0.5762ins137 mutation with the specified antisense oligonucleotides.

FIG. 6 is a diagram of an in vitro assay based on the ATM downstream signaling pathway (Menolfi and Zha, Genome Instability & Disease (2019), the contents of which are hereby incorporated by reference in their entirety. Assessment of phosphorylation state of downstream ATM targets p53 and Kap1 allows for functional evaluation of ATM kinase activity in intact patient cells. “γH2AX” denotes a variant of the histone H2A protein family that is a marker of double-stranded DNA breaks and is phosphorylated by ATM.

FIGS. 7A and 7B illustrate results of the assay depicted in FIG. 6. FIG. 7A is a Western blot image showing phosphorylated Kap1 (pKap1) and phosphorylated p53 (p-p53) 48 hours after transfection of the indicated ASOs targeting the NM_000051.3(ATM):c0.5762ins137 mutation and irradiation. “Control” denotes fibroblasts from an individual who is a carrier of an ATM mutation. “772” denotes a scrambled antisense oligonucleotide. “-” indicates that the cells were not irradiated. “+” indicates that the cells were exposed to 10 Gy of radiation. FIG. 7B is a graph illustrating the normalized level of pKap1 and p-p53 detected in the cells relative to the pKap1 and p-p53 levels detected in irradiated control cells.

FIG. 8 is a diagram illustrating the strategy used for rescuing abnormal splicing induced by the NM_000051.3(ATM):c0.7865C>T mutation, employing steric blockade by a splice-switching antisense oligonucleotide.

FIG. 9 is a diagram showing the first-round design of antisense oligonucleotides (ASOs) to block abnormal splicing induced by the NM_000051.3(ATM):c0.7865C>T mutation. Candidates AT001-AT011 are depicted. A twelfth candidate (AT012, not pictured) targets a downstream intronic splice silencer for the native exon 53 splice site (inhibition of this silencer would be predicted to strengthen normal splicing). Figure discloses SEQ ID NOS 63-64, respectively, in order of appearance.

FIGS. 10A and 10B illustrate ATM splicing modulation by candidate ASOs targeting NM_000051.3(ATM):c0.7865C>T. FIG. 10A is a gel image showing an assessment of ATM splicing modulation by candidate ASOs via RT-PCR. Patient fibroblasts treated with candidates AT001-AT012 display an upper band representing wild-type full-length exon 53 splice product. This band is notably absent in untreated (or mock-transfected, or transfected with a scrambled ASO) patient fibroblasts. FIG. 10B is a graph showing quantification of % wild-type splicing by candidate ASOs. Candidate AT008 shows robust activity with recovery of wild-type splicing up to 50% of total transcripts.

FIG. 11 is a schematic illustration of ASOs, designed for NM_000051.3(ATM):c0.7865C>T in the fine-tuning round. The shading of the ASOs indicates their efficacy in patient fibroblast splicing assays with the lightest shading associated with lower efficacy and the black shading indicating highest efficacy. Several ASOs, including AT026 (21 nt), display efficacy similar to AT008 (22 nt). Figure discloses SEQ ID NOS 63-64, respectively, in order of appearance.

FIG. 12 is a gel image showing dose-response curves of ATM splicing to AT008 or AT026 as assessed by RT-PCR in fibroblasts derived from an A-T patient with NM_000051.3(ATM):c0.7865C>T. Normally-spliced exon 53 products are observed at doses down to 1 nM for both antisense oligonucleotides.

FIGS. 13A-13D illustrate functional restoration of ATM signaling in fibroblasts derived from an A-T patient having the NM_000051.3(ATM):c0.7865C>T mutation in the ATM gene. FIG. 13A comprises images of patient fibroblasts that demonstrate activation and nuclear localization of phosphorylated p53 (p-p53) and phosphorylated Kap1 (pKap1) 90 minutes after 1.5 Gy irradiation. The cells shown in the two columns to the right were treated with indicated ASOs. FIG. 13B is a Western blot image showing p-p53 and pKap1 in fibroblast cells transfected (400 nM) with antisense oligonucleotides and irradiation (10 Gy). “Control” denotes fibroblasts derived from an individual without A-T but a heterozygous carrier of NM_000051.3(ATM):c0.7865C>T. “A-T patient without c0.7865C>T” denotes fibroblasts derived from an A-T patient without

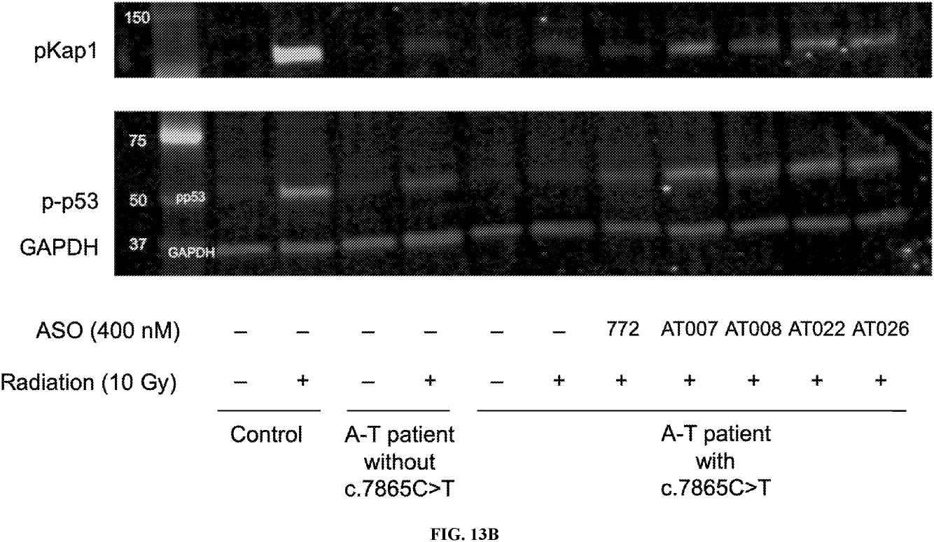

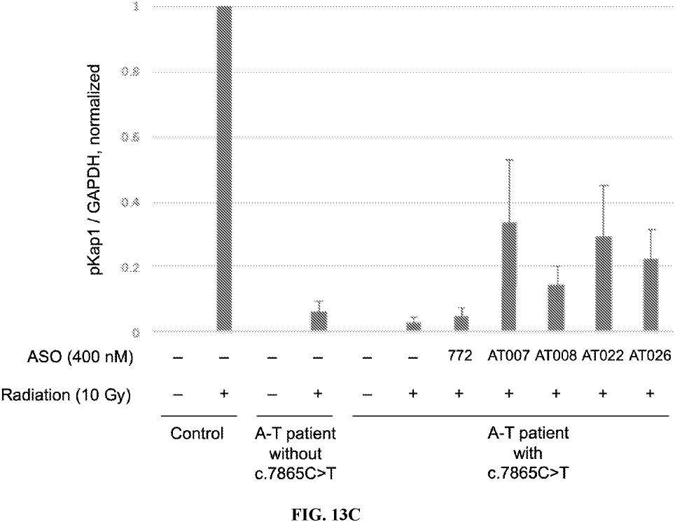

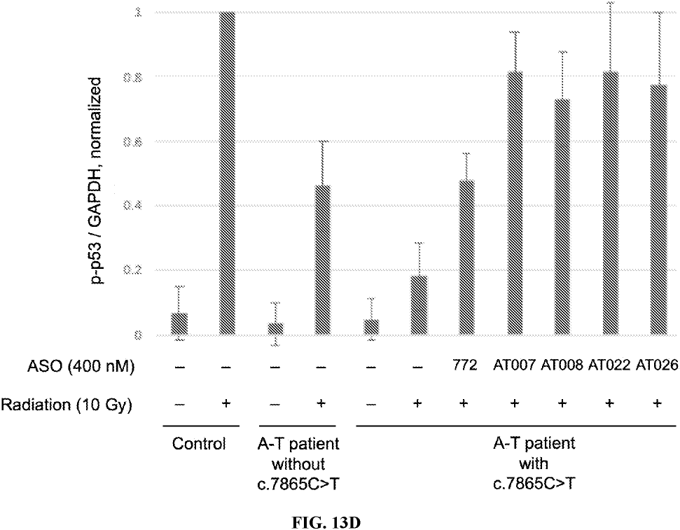

NM_000051.3(ATM):c0.7865C>T. “A-T patient with c0.7865C>T” denotes fibroblasts derived from an A-T patient with NM_000051.3(ATM):c0.7865C>T. FIG. 13C is a graph quantifying pKap1 levels in FIG. 13B. pKap1 levels were first normalized by GAPDH levels and compared to the level of the untreated, unirradiated control fibroblasts. Error bars denote 95% confidence intervals. FIG. 13D is a graph quantifying p-p53 levels in FIG. 13B. p-p53 levels were first normalized by GAPDH levels, and compared to the level of the untreated, unirradiated control fibroblasts. Error bars denote 95% confidence intervals.

### DETAILED DESCRIPTION OF THE INVENTION

As described below, the present invention features antisense oligonucleotides that block abnormal splicing of an ATM gene comprising a mutation associated with A-T, and methods of using such oligonucleotides in treating A-T.

The invention is based, at least in part, on the discovery of antisense oligonucleotides that restore wild-type splicing of an ATM gene having a mutation that affects splicing. For example, the compositions and methods can be used to recover wild-type splicing of an ATM allele having a mutation that alters splicing.

### Ataxia Telangiectasia (A-T)

Ataxia Telangiectasia is an autosomal recessive disease characterized by progressive neurological degeneration. As shown in Tables 3 and 4, the onset of symptoms occurs early in life.

TABLE 3

Clinical and laboratory features of the patients with ataxia telangiectasia (continuous variable)

| Variables | Mean±standard deviation |
|---|---|
| Age of symptom onset (mon) | 15.40±1.09 |
| Age of diagnosis (mon) | 73.61±4.11 |
| White blood cell (/mm) | 8811.36±401.18 |
| Absolute lymphocyte count (/mm³) | 2892.42±194.53 |
| IgG (mg/dL) | 971.62±44.01 |
| IgG₂ (mg/dL) | 145.11±58.33 |
| IgM (mg/dL) | 219.90±15.49 |
| IgA (mg/dL) | 63.04±7.99 |
| IgE (kU/L) | 29.18±11.67 |
| CD3 (/mm³) | 1702.43±164.73 |
| CD4 (/mm³) | 947.25±135.43 |
| CD8 (/mm³) | 769.75±77.97 |
| CD19 (/mm³) | 370.58±67.31 |
| CD16+56+ (/mm³) | 556.14±60.35 |

Ig: imminoglobulin: CD: cluster of differentiation.

(Akturk et al., World J Pediatr, 13(5):465-71 (2017)).

TABLE 4

Clinical symptoms of 104 ataxia-telangiectasia patients

| Symptom | Patients Affected, No. | Age of Onset, Years, Median (range) |
|---|---|---|
| Ataxia | 104 | 3 (1-7) |
| Speech disorder | 22 | 10 (8-18) |
| Eye movement disability | 73/93^{∗} | 8 (7-18) |
| Choreoathetotic movement | 81/93^{∗} | 6 (4-8) |
| Seizure | 9 | |
| Mental retardation | 10 | |
| Growth retardation | 39 | |

^{∗} Data were lacking for this symptom in 11 patients.
^{†} Age of onset for this symptom could not be determined.

(Moin et al., Pediatr Neurol., 37(1):21-8 (2007)).

By age 2, clinical signs of A-T may include delayed walking, wobbly gait, issues standing or sitting still, and truncal sway. Cerebellar atrophy may be evident in MRI examination and other neuropathologic evidence of cerebellar degeneration may be observed by age 3 or 4. These cerebellar changes can become more advanced and severe with age along with additional spinal cord and brainstem involvement.

By age 10, cerebellar atrophy has progressed, and degenerative changes of Clarke’s columns and inferior olives may be observed. At about this age, patients typically exhibit loss of myelinated fibers in posterior columns, dorsal root ganglia (DRG) degeneration, and muscle atrophy.

Neurodegeneration continues through adulthood including the progression and expansion of symptoms that began at an early age. Throughout adulthood, subjects afflicted with A-T can expect axonal degeneration of peripheral nerves and new spinal lesions (e.g., gliosis of the posterior columns, loss of myelinated fibers in the spinocerebellar and corticospinal tracts, and anterior horn cell degeneration). In some cases, subjects may have variable brainstem changes, such as depigmentation of locus caeruleus, degenerative changes in the substantia nigra, and degeneration of cranial nerves.

Many mutations have been associated with A-T to-date. A subset of the mutations causes aberrant splicing that leads to premature truncation of the ATM protein. As important domains (i.e., kinase domains) reside towards the carboxy terminus of the protein, premature truncation can effectively eliminate or impair the function of the protein. The present invention provides compositions and methods that restore wild-type splicing in patients and cells that have a mutation in the ATM gene that causes aberrant splicing. For example, the invention provides antisense oligonucleotides that can be used therapeutically to treat A-T. In some embodiments, these antisense oligonucleotides are designed specifically to treat A-T associated with ATM(NM_000051.3):c0.5762_5763insNG_009830.1:g0.91138_91274 (alternate: NM_000051.3(ATM):c0.5762ins137), a mutation that is found in 18% of patients with A-T in the United Kingdom. The invention also provides antisense oligonucleotides that can be used therapeutically to treat A-T due to NM_000051.3(ATM):c0.7865C>T, a mutation that has been identified in approximately 4.5% of A-T patients in Turkey (Teraoka et al., Am. J. Hum. Genet. 64:17-1631 (1999); Eng et al., Human Mutation 23:67-76 (2004), the contents of which are hereby incorporated by reference in their entirety).

### NM_000051.3(ATM):c0.5762ins137

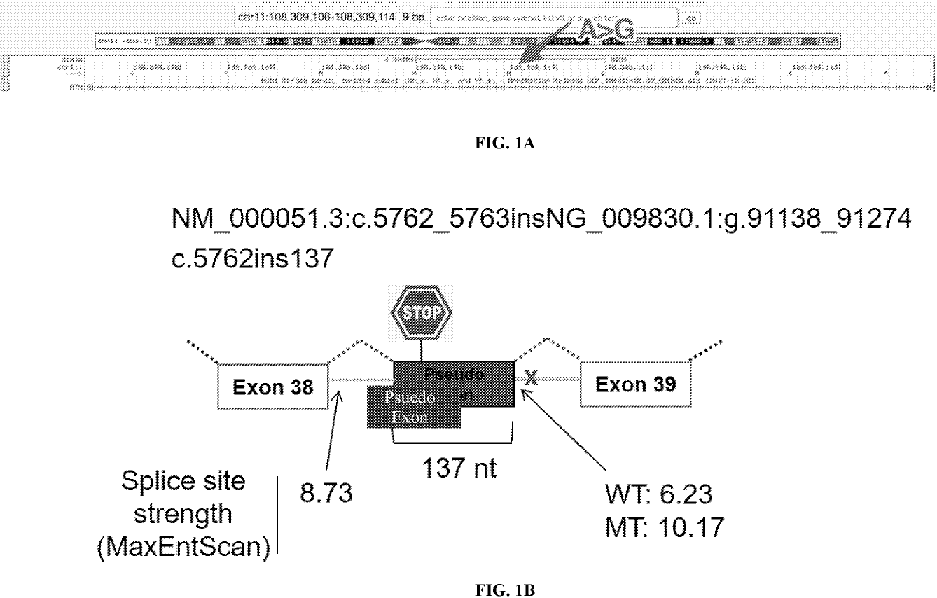

Mutations in the ATM gene cause aberrant splicing and loss of ATM kinase activity. One such mutation observed in a subject having A-T was the NM_000051.3(ATM):c0.5762ins137 mutation (also referred to as ATM(NM_000051.3):c0.5762_5763insNG_009830.1:g0.91138_91274). This is a known mutation associated with A-T that has a genomic coordinate of chr11:108309110 A>G (hg38) (FIG. 1A). This A-to-G point mutation affects splicing by causing an insertion of a 137 nucleotide pseudo-exon between exons 38 and 39. The predicted splice site strength is greater for the NM_000051.3(ATM):c0.5762ins137 mutation than for the wild-type splice site at the 3′ terminus of exon 38. This pseudo-exon comprises a premature stop codon, which causes truncation of the ATM protein and loss of a portion of the kinase domain (FIG. 1B).

The frequency of A-T is approximately one case per 40,000 to 100,000 births, which equates to 40 to 100 new cases in the U.S. and 7 to 17 new cases in the U.K. per year. The NM_000051.3(ATM):c0.5762ins137 mutation associated was observed in eight subjects of a U.S. cohort (n = 115), and this frequency (approximately 0.07) suggests approximately 3 to 7 new U.S. cases per year are subjects carrying this mutation. In the U.K., the frequency of the mutation is approximately 0.18 (Table 5), which suggests approximately 1 to 3 new cases of A-T per year in the U.K. in which the patient carries the NM_000051.3(ATM):c0.5762ins137 mutation.

TABLE 5

ATM mutations in different populations

| Population (Haplotype) | Mutation | Frequency (% alleles) |
|---|---|---|
| Amish Polish | 1563delAG | >99 |
| A | IVS53-2A>C | 15 |
| B | 6095G>A | 8 |
| C | 7010delGT | 6 |
| D | 5932G>T | 10 |
| E | 1563delAG | 6 |
| Norwegian | | |
| A | 3245ATC>TGAT | 55 |
| United Kingdom^{∗} | | |
| FM7 | 5762ins137 | 18 |
| FM10 | 7636del19 | 15 |

Perlman et al., 2012.

The NM_000051.3(ATM):c0.5762ins137 mutation is pathogenic. Five submissions to the ClinVar database classify this mutation as pathogenic (www.ncbi.nlm.nih.gov/clinvar/RCV000003157/), and the GnomAD frequency (3.27 × 10⁵) is compatible with a pathogenic variant.

### NM_000051.3(ATM):c0.7865C>T

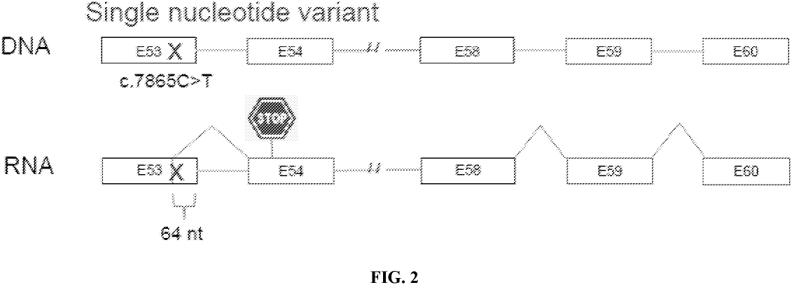

Another mutation observed in a subject diagnosed with A-T was the NM_000051.3(ATM):c0.7865C>T mutation. NM_000051.3(ATM):c.7865C>T is a recurrent Turkish missense mutation (A2622V) found on the TAT[C] haplotype and has been identified in approximately 4.5% of Turkish patients (Teraoka et al., Am. J. Hum. Genet. 64:17-1631 (1999); Eng et al., Human Mutation 23:67-76 (2004)). This mutation creates a novel splice site that truncates 64 nucleotides from the 3′ end of exon 53 (FIG. 2, see the Maternal allele). This truncation cases a shift in the reading frame of the transcript and, consequently, a premature stop codon in exon 54. The premature stop results in the loss of critical residues from the highly conserved PI3K/PI4K kinase domain of the ATM protein.

### Antisense Oligonucleotide-Mediated Recovery of Wild-Type Splicing of the ATM Gene

Antisense oligonucleotides are provided that restore wild-type splicing of an ATM gene having a mutation that leads to aberrant splicing of the gene. In one embodiment, an oligonucleotide or nucleobase oligomer of the invention comprises 2′-modified oligonucleotides where some or all internucleotide linkages are modified to phosphorothioates or phosphodiester (PO). In some embodiments, the presence of methylphosphonate modifications increases the affinity of the oligonucleotide for its target RNA and thus reduces the IC50. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Pat. Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′, or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. In some embodiments, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. One such nucleobase oligomer, is referred to as a peptide nucleic acid (PNA). In PNA compounds, the sugar-backbone of an oligonucleotide is replaced with an amide containing backbone, in particular, an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular --CH₂--NH--O--CH₂--, --CH₂--N(CH₃)--O--CH₂-- (known as a methylene (methylimino) or MMI backbone), --CH₂--O--N(CH₃)-CH₂-, --CH₂--N(CH₃)--N(CH₃)--CH.₂--, and --O--N(CH₃)--CH₂--CH₂--. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O--, S--, or N-alkyl; O--, S--, or N-alkenyl; O--, S-- or N--alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH2)nNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C1 to C10 lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Exemplary 2′ modifications are 2′-O-methyl and 2′-methoxyethoxy (2′-O--CH₂CH₂OCH₃, also known as 2′-O-methoxyethyl (2′-MOE). Other desirable modifications include 2′-dimethylaminoethoxyethoxy and 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂), 2′-fluoro (2′-F), and 2′-acetamide. Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. In some embodiments, a nucleobase oligomer comprises a locked nucleic acid (LNA). Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G) and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl, and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo (e.g., 5-bromo), 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-78) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-60, 1994), a thioether, e.g., hexyl-S-tritylthiol (Manoharan et al., Ann. N.Y. Acad. Sci., 660:306-09, 1992; Manoharan et al., Bioorg. Med. Chem. Let., 3:2765-70, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-18, 1991; Kabanov et al., FEBS Lett., 259:327-30, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-54, 1995; Shea et al., Nucl. Acids Res., 18:3777-83, 1990), a polyamine or a polyethylene glycol chain (Manoharan et al., Nucleosides & Nucleotides, 14:969-73, 1995), or adamantane acetic acid (Manoharan et al., Tetrahedron Lett., 36:3651-54, 1995), a palmityl moiety (Mishra et al., Biochim. Biophys. Acta, 1264:229-37, 1995), or an octadecylamine or hexylamino-carbonyl-oxycholesterol moiety (Crooke et al., J. Pharmacol. Exp. Ther., 277:923-37, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241; 5,391,723; 5,414,077; 5,416,203; 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated, or otherwise associated with other molecules, molecule structures or mixtures of compounds, as for example, liposomes, receptor targeted molecules, oral, rectal, topical, or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The nucleobase oligomers of the invention encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “pharmaceutically acceptable salts” refers to salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., Berge et al., J. Pharma Sci., 66:1-19, 1977). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides and other nucleobase oligomers, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

The present invention also includes pharmaceutical compositions and formulations that include the nucleobase oligomers of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral, or parenteral. Parenteral administration includes intravenous, intraarterial, subcutaneous, intraperitoneal, or intramuscular injection or infusion; or intracranial, e.g., intrathecal or intraventricular, administration.

### Screening for Agents to Recover Wild-Type Splicing of a Gene

In one aspect, the invention provides methods of screening for antisense oligonucleotides that restore wild-type splicing in cells that have a mutation that leads to aberrant splicing. Thus, in various embodiments, the method will screen for an antisense oligonucleotide that restores wild-type splicing in cells having a mutated splice site, a mutation that generates a novel splice site, or a mutation in a splicing regulatory element (e.g., an ESE or an ESS).

Numerous methods are also available for generating random or directed synthesis (e.g., semi-synthesis or total synthesis) of oligonucleotides. The screening methods presented herein are amendable to a high throughput format.

In some embodiments, antisense oligonucleotides are synthesized that are complementary to a nucleic acid sequence in an ATM allele that contains a mutation that impairs wild-type splicing or causes aberrant splicing. These antisense oligonucleotides can be transfected into cells (e.g., a fibroblast cell) comprising an ATM allele having a splicing mutation. Transcripts of the allele can be analyzed to determine if the ATM allele is correctly spliced. Methods of detecting and characterizing mRNA transcripts are known in the art, such as oligonucleotide microarray assays, quantitative RT-PCR, Northern analysis, and multiplex bead-based assays. In some embodiments, RT-PCR can be used to amplify the transcripts, and the resulting amplification products can be visualized on a gel or by any other means known in the art.

An antisense oligonucleotide identified as being able to restore wild-type splicing (i.e., a lead antisense oligonucleotide) can be optimized to increase the percentage and/or efficiency of wild-type splicing in a cell. For example, in some embodiments, additional antisense oligonucleotides (“optimized antisense oligonucleotides”) that have substantial sequence identity to the lead antisense oligonucleotide are generated. In some embodiments, the optimized antisense nucleotides include modified nucleobases or backbones that enhance binding to RNA transcripts. In other embodiments, the optimized antisense oligonucleotides differ in length from the lead antisense oligonucleotide.

In some embodiments, an antisense oligonucleotide’s ability to restore wild-type ATM splicing can be assessed on the presence of downstream effects that are dependent on a mature, wild-type ATM protein. For example, detection of phosphorylated Kap1 or p53, after irradiating a cell suggests that the spliced ATM transcript was translated into a functional ATM kinase (see Menolfi and Zha, Genome Instability & Disease (2019), the contents of which are hereby incorporated by reference in their entirety).

### Restoring Wild-Type Splicing in a Cell Harboring a Splicing Mutation in the ATM Gene

There are several known mutations that disrupt wild-type splicing of ATM. Provided herein are methods of restoring wild-type splicing in a cell that has a mutation in the ATM gene that results in aberrant splicing. In some embodiments, the methods comprise contacting a cell having a mutation in the ATM gene that causes aberrant splicing with one or more antisense oligonucleotides described herein. In some embodiments, the antisense oligonucleotide can be an antisense oligonucleotide in Table 1 or 2. In some embodiments, the antisense oligonucleotide has one or modification described herein.

Contacting the cell with an antisense oligonucleotide of the present invention can result in about 10%, about 20%, about 30%, about 40%, about 50%, about 60%, about 70%, about 80%, about 90%, or about 100% of all ATM transcripts being wild-type. In some embodiments, contacting the cell with an antisense oligonucleotide of the present invention results in 30% or greater, 50% or great, 75% or greater, or even 90% or greater increase in wild-type splicing compared to an untreated cell.

In some embodiments, the cell is a cell whose function is impaired by A-T (e.g., neuron, immune cell, and/or hepatocyte). In some embodiments, the contacting is in vitro. In some embodiments, the contacting is in vivo. In some embodiments, the cell comprises the NM_000051.3(ATM):c0.5762ins137 mutation in the ATM gene. In some embodiments, the cell comprises the NM_000051.3(ATM):c0.7865C>T mutation in the ATM gene. The antisense oligonucleotide can be designed to hybridize with a splice acceptor or a splice donor or a splicing regulatory element. In some embodiments, the cell is contacted by a combination of antisense oligonucleotides, wherein one antisense oligonucleotide of the combination is designed to hybridize to a splice acceptor or donor site or a splicing regulatory element and at least one additional antisense oligonucleotide of the combination is designed to hybridize with a different splice site or splicing regulatory element.

### Methods of Treating A-T

Aspects of the present invention provide methods of treating a subject having or suspected of having A-T by administering antisense oligonucleotides of the present invention. In some embodiments, methods are provided for treating or preventing the disease or symptoms thereof that comprise administering a therapeutically effective amount of a pharmaceutical composition comprising the antisense oligonucleotides disclosed herein to a subject (e.g., a mammal such as a human). Administering the therapeutically effective amount of an antisense oligonucleotide as described herein comprises administering an amount sufficient to treat the disease or disorder or symptom thereof, under conditions such that the disease or disorder is treated.

Administration of antisense oligonucleotides to a subject having or suspected of having A-T can be targeted or systemic. Generally, the antisense oligonucleotide will be administered in a pharmaceutical composition as described above.

In some embodiments, a composition comprising the antisense oligonucleotides of the present invention is conveniently presented in unit dosage form and is prepared by any method well known in the art. The dosage of the administered antisense oligonucleotide depends on a number of factors, including the size and health of the individual patient. For any particular subject, the specific dosage regimes should be adjusted over time according to the individual need and the professional judgment of the person administering or supervising the administration of the compositions. The amount of active ingredient that can be combined with a carrier material to produce a single dosage form will vary depending upon not only the host being treated but also the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, from about 5 per cent to about 70 per cent, or from about 10 per cent to about 30 per cent.

Additional suitable carriers and their formulations are described, for example, in the most recent edition of Remington’s Pharmaceutical Sciences by E. W. Martin. The amount of the therapeutic agent to be administered varies depending upon the manner and mode of administration, the age and disease status (e.g., the extent of hearing loss present prior to treatment).

Compositions are administered at a dosage that controls the clinical or physiological symptoms of the disease or condition, as may in some cases be determined by a diagnostic method known to one skilled in the art, or using any assay that measures the biological activity of an antisense oligonucleotide or composition comprising an antisense oligonucleotide, or a combination thereof.

Therapeutic compounds and therapeutic combinations are administered in an effective amount. In certain embodiments, compositions, such as those described herein, are administered at dosage levels of about 0.0001 to 1.0 g once per day (or multiple doses per day in divided doses). In certain embodiments, an antisense oligonucleotide or composition as described herein is administered at a dosage between 0.1 mg/day and 100 mg/day and the upper end of the range is any amount between 1 mg/day and 1000 mg/day (e.g., 5 mg/day and 100 mg/day, 150 mg/day and 500 mg/day). In other embodiments, a compound or composition as herein is administered at a dosage range in which the low end of the range is any amount between 0.1 mg/kg/day and 50 mg/kg/day and the upper end of the range is any amount between 1 mg/kg/day and 100 mg/kg/day (e.g., 0.5 mg/kg/day and 2 mg/kg/day, 5 mg/kg/day and 20 mg/kg/day). In some embodiments, a composition of the invention is administered at a dosage of about 10 mg/dose, about 20 mg/dose, about 30 mg/dose, about 40 mg/dose, about 50 mg/dose, about 60 mg/dose, about 70 mg/dose, about 80 mg/dose, about 90 mg/dose, about 100 mg/dose, about 110 mg/dose, about 120 mg/dose, about 130 mg/dose, about 140 mg/dose, about 150 mg/dose, about 160 mg/dose, about 170 mg/dose, about 180 mg/dose, about 190 mg/dose, or even about 200 mg/dose. In some embodiments, a composition of the invention is administered at a dosage of between 1 and 10 mg/dose. For example, in some embodiments, a composition of the invention is administered at a dosage of about 1, 2, 3, 4, 5, 6, 7, 8, 9, or 10 mg/dose.

In some embodiments, administration may not be daily, but rather once every 2, 4, 6, 8, or 10 weeks. For example, in administering antisense oligonucleotides to children, the dosage regimen may be between 1 and 50 mg every two weeks for 8 to 10 weeks followed by 1 to 100 mg every four weeks for 3 to 4 weeks, followed by every 8 weeks for 16 to 48 weeks. The dosing interval can be adjusted according to the needs of individual patients.

### Kits

The invention provides kits for the treatment or prevention of A-T or symptoms thereof. In one embodiment, the kit includes a pharmaceutical pack comprising an effective amount of one or more antisense oligonucleotides. In some embodiments, the compositions are in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions or combinations thereof are provided together with instructions for administering them to a subject having or at risk of developing A-T. The instructions will generally include information about the use of the compounds for the treatment or prevention of A-T. In other embodiments, the instructions include at least one of the following: description of the compound or combination of compounds: dosage schedule and administration for treatment of A-T or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention. All publications cited herein are incorporated by reference for the purposes or subject matter referenced herein.

### EXAMPLES

### Example 1: Identifying a Splicing Mutation in a Subset of A-T Patients

Ataxia telangiectasia (A-T) is an autosomal recessive disease caused by mutations in the ATM kinase gene. To identify mutations in ATM associated with A-T, whole genome sequencing was performed on samples from subsets of A-T patients residing in the US (n = 115). Sequencing data was analyzed to identify mutations predicted to affect splicing of the ATM gene; for example, mutations in canonical splicing sites that flank the gene’s exons, mutations in splicing regulatory elements (SREs) (e.g., exonic splicing enhancers (ESEs). The functions of detected sequence variants were predicted using Variant Effect Predictor (VEP). The ClinVar database was also accessed to ascertain other information related to detected sequence variants.

An adenine to guanine nucleotide substitution, NM_000051.3(ATM):c0.5762ins137 was observed the ATM gene on chromosome 11 (FIG. 1A). As explain supra, this mutation causes aberrant splicing in which a 137 psuedo-exon with a premature stop codon is incorporated into the ATM transcript (FIG. 1B).

### Example 2: Antisense Oligonucleotide Design and Screening

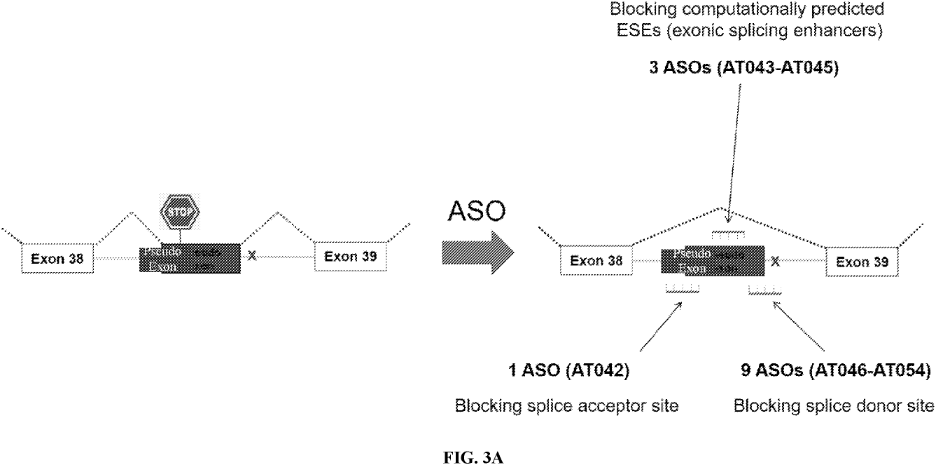

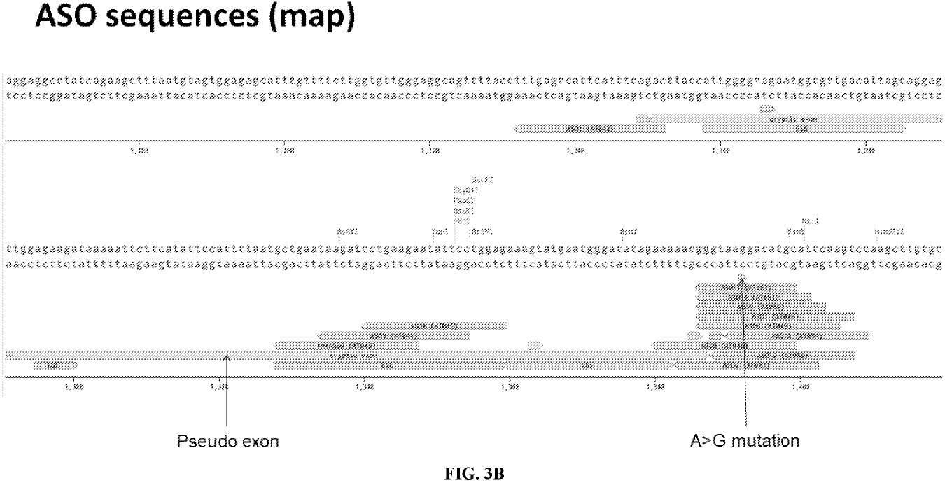

It was hypothesized that by hybridizing complementary ASOs to splicing regulatory regions in the pseudo-exon, wild-type splicing could be restored. Referring to FIGS. 3A and 3B, oligonucleotides were designed to bind to three different regions of the pseudo-exon: the splice acceptor site (AT042), a computationally predicted exonic splicing enhancer (ESE) (AT043-045), and the splice donor site (AT046-054). The sequences for the oligonucleotides are provided in Table 1.

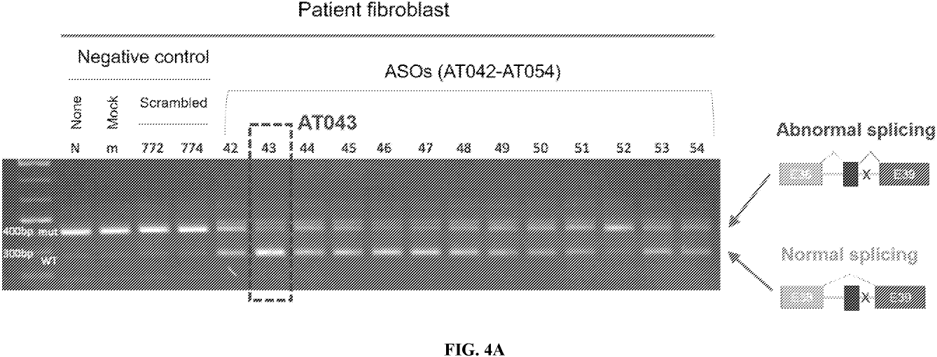

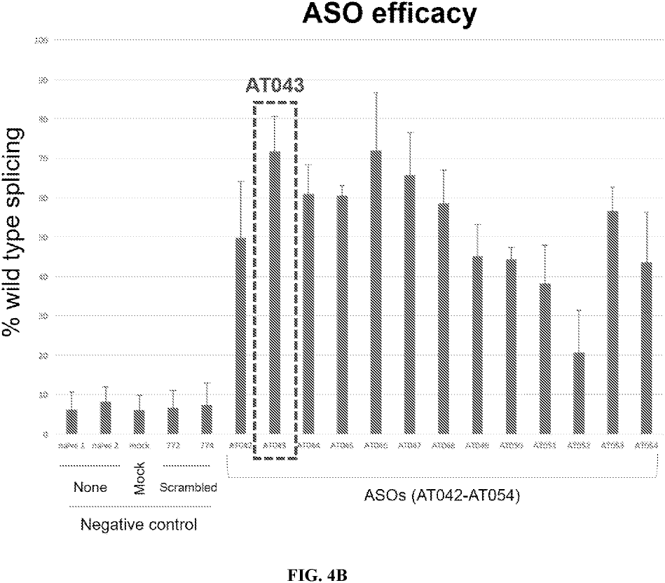

Once designed, the oligonucleotides were screened for activity. Briefly, a cell model of A-T comprised of cultured fibroblasts from an A-T patient with the NM_000051.3(ATM):c0.5762ins137 mutation (heterozygous) were transfected with 200 nM of each of the oligonucleotides listed in Table 1. Cells were cultured for 48 hours with each oligonucleotide before total RNA was prepared. To determine if contacting the cells with the oligonucleotides resulted in improved wild-type splicing, an allele-specific RT-PCR assay was used to visualize splicing patterns. Referring to FIG. 4A, abnormal splicing is observed in all samples tested, but several oligonucleotides promoted wild-type splicing. Greater than 50% wild-type splicing was observed in more than half of the cultures transfected with an antisense oligonucleotide, with oligonucleotide AT043 promoting greater than 70% wild-type splicing. (FIG. 4B). This oligonucleotide was selected for optimization.

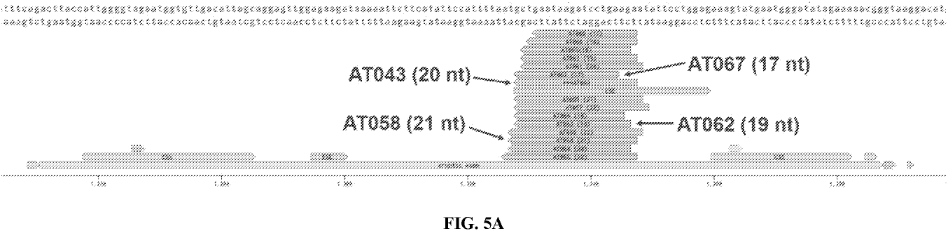

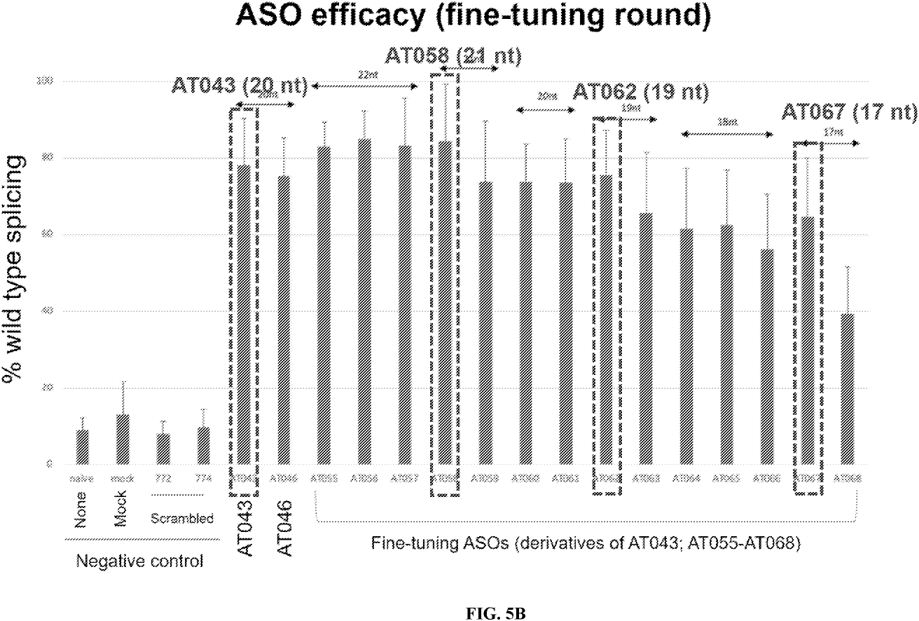

Optimizing oligonucleotide AT043 consisted of generating overlapping oligonucleotides (AT055 to AT067) that comprise a majority of the nucleotide sequence of AT043. As shown in FIG. 5A, the candidate optimized antisense oligonucleotides differ in length, starting position or ending position from AT043. The candidate optimized antisense oligonucleotides are also provided in Table 1.

To determine the extent to which these antisense oligonucleotides restored wild-type splicing, cell cultures were transfected with these antisense oligonucleotides. All of the ASOs restored wild-type splicing, with many restoring greater than 60% wild-type splicing (FIG. 5B).

### Example 3: Functional Assay of Antisense Oligonucleotides

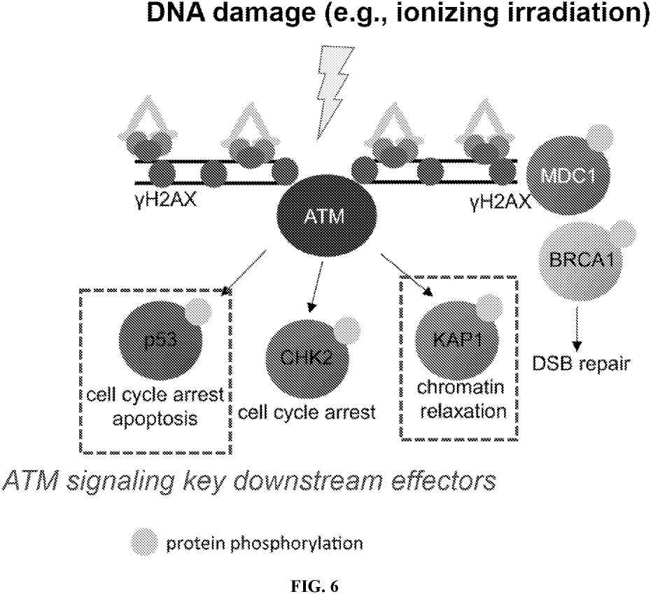

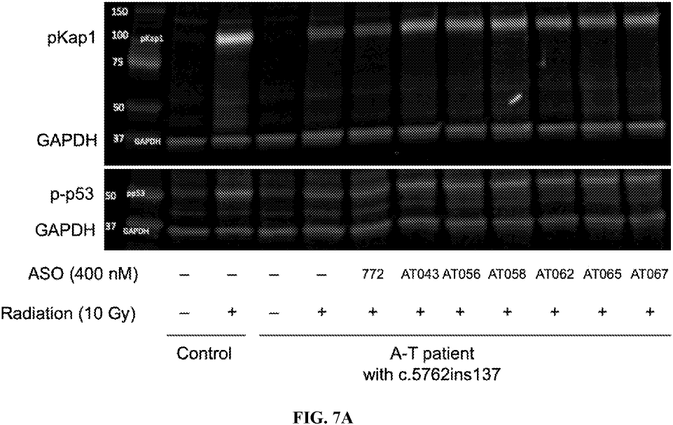

ATM phosphorylates targets in cell cycle checkpoint signaling pathways required for responding to DNA damage. An assay was employed that leveraged this ATM function to determine if cells carrying the mutant (NM_000051.3(ATM):c0.5762ins137) ATM allele and transfected with the antisense oligonucleotides AT043, AT056, AT058, AT062, AT065, and AT067 functioned properly after exposure to ionizing irradiation (i.e., by phosphorylating p53, CHK2, and Kap1 (FIG. 6 (adapted from Menolfi and Zha, Genome Instab. Dis. (2019) dx.doi.org/10.1007/s42764-019-00003-9)). After transfecting the cells and exposing them to 1.5 Gy of radiation, the cell cultures were incubated and then assayed for target phosphorylation. Specifically, cell cultures were homogenized and treated with primary antibodies that specifically bind phosphorylated Kap1 or p53 and antibodies that specifically bind GADPH, which served as a loading control.

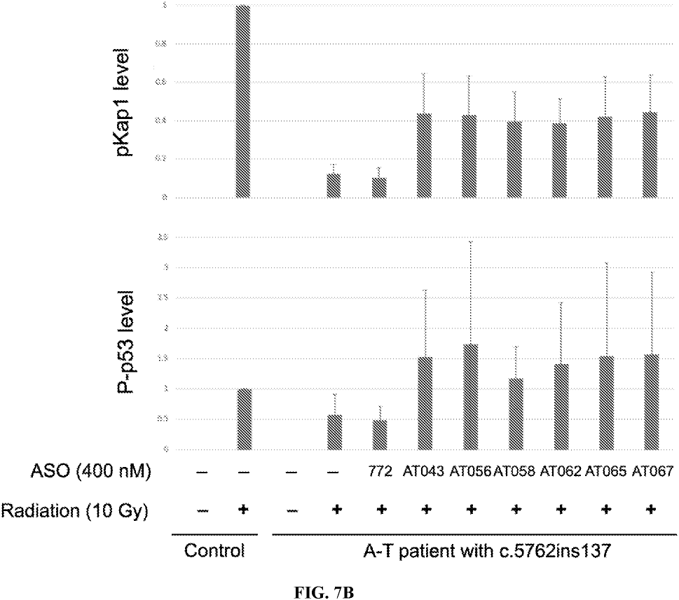

Referring to FIGS. 7A and 7B, each cell culture transfected with the indicated oligonucleotides showed phosphorylation of Kap1 and p53, which indicated wild-type splicing of the ATM gene. Fibroblasts derived from a heterozygous ATM mutation carrier is used as a positive control. A strong phosphorylation signal for pKap1 and p53 in control irradiated fibroblasts compared to non-irradiated fibroblasts. Base levels of pKap and p53 phosphorylation after irradiation of A-T patient fibroblasts heterozygous for the NM_000051.3(ATM):c0.5762ins137 mutation show a much lower level of phosphorylation compared to the positive control. Fibroblasts derived from an A-T patient with the NM_000051.3(ATM):c0.5762ins137 mutation exhibit recovered wild-type splicing after treatment with antisense oligonucleotides AT056, AT058, AT062, AT065, and AT067.

### Example 4: Identification and Characterization of Sequence Variants in a Subject Presenting with A-T Symptoms and Treatment Design

A patient, was notable for abnormal T-cell receptor excision circle at newborn screening, which raised the possibility of severe combined immunodeficiency (SCID). While her immunologic workup was negative, two different mutations were identified by whole exosome sequencing (WES). One of the mutations detected, c0.8585-13_8598de127, disrupts a splice acceptor site in exon 59 and removes residues from the highly conserved PI3K/PI4K kinase domain of the ATM protein. The other mutation, NM_000051.3(ATM):c0.7865C>T, creates a novel splice site that truncates 64 nucleotides at the 3′ end of exon 53, thereby shifting the reading frame of the transcript and generating a premature stop codon in exon 54. FIG. 2. Being compound heterozygous for these mutations results in the classical form of ataxia telangiectasia (A-T).

Consistent with classical A-T, the patient sat at 5 months, pulled to stand at 16 months, and her first independent steps were taken at 24 months. The patient also exhibited low muscle tone, truncal swaying when sitting, fatigue at the end of exam, and a limited vocabulary of only 10-15 words.

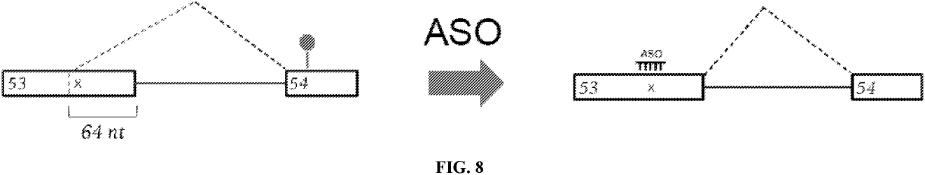

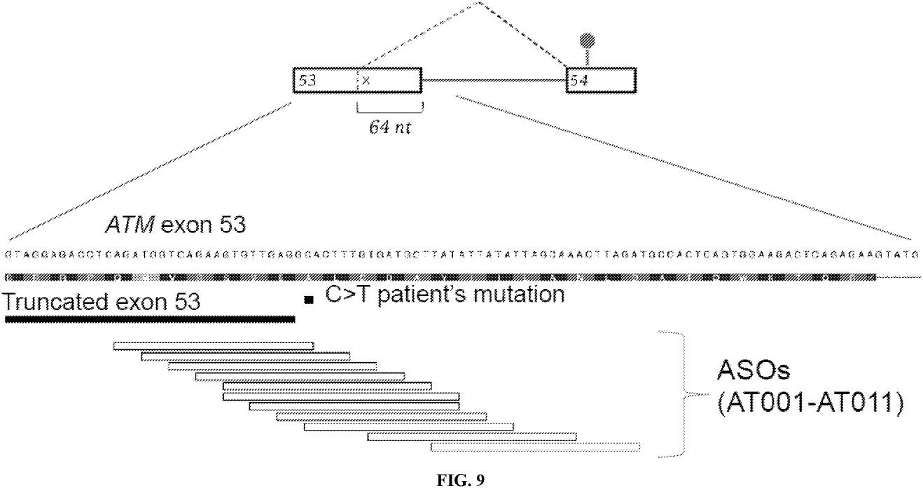

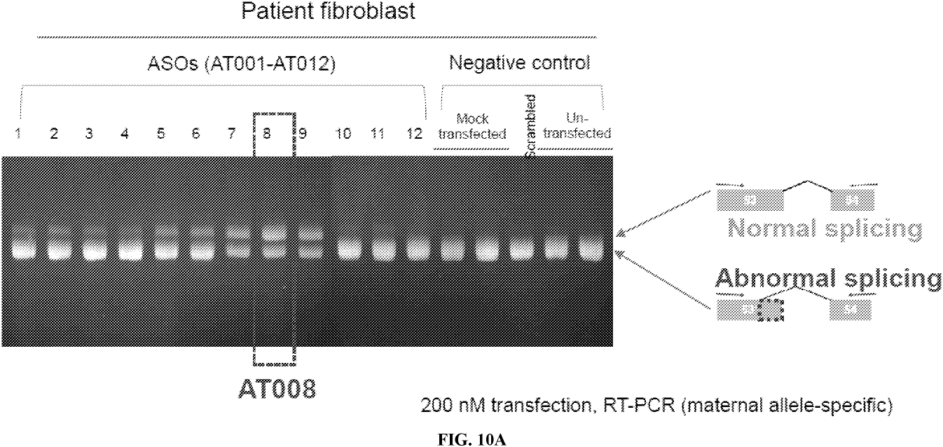

Due to the creation of a novel splice site by the maternally inherited NM_000051.3(ATM):c0.7865C>T mutation, it was hypothesized that antisense oligonucleotides could be designed to effectively block abnormal splicing induced by the mutation (FIG. 8). To test this hypothesis, 11 antisense oligonucleotides (AT001-AT011) were designed (FIG. 9). A twelfth candidate (AT012) was designed to target a downstream intronic splice silencer for the native exon 53 splice site (see Table 2). Inhibition of the silencer was predicted to strengthen normal splicing.

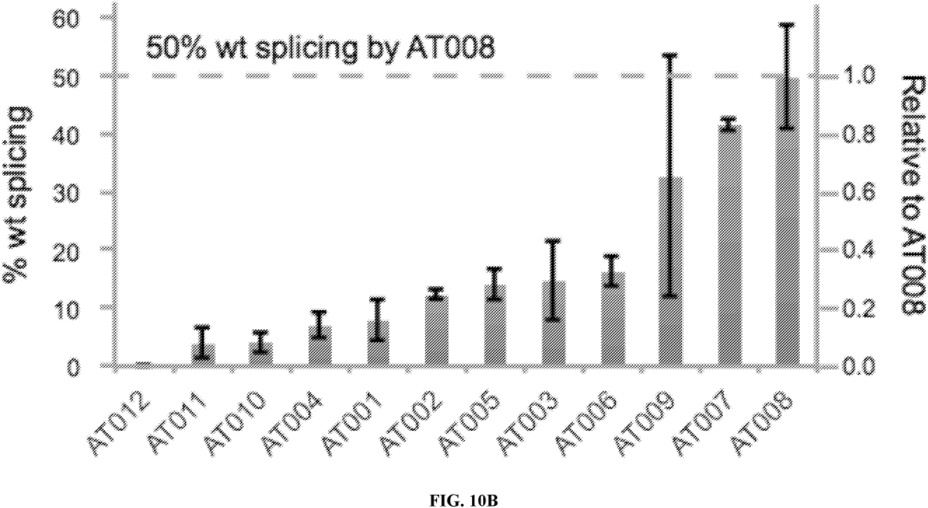

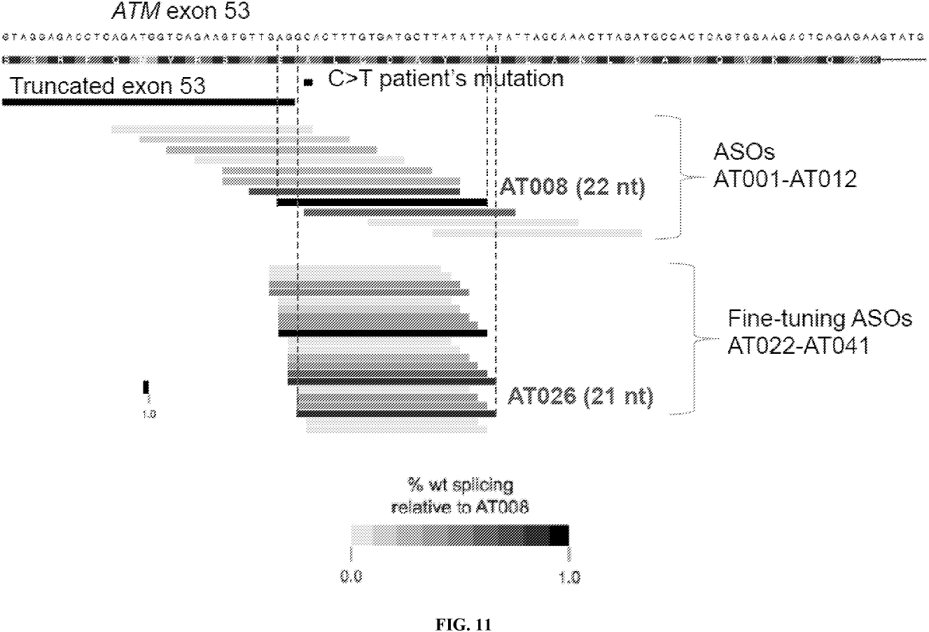

The candidate antisense oligonucleotides were screened in fibroblasts derived from the patient as described in Example 2 above. These fibroblasts, when treated with candidates AT001 to AT012 display an upper band representing wild-type full-length exon 53 splice product (FIG. 10A). Notably, the band demonstrating wild-type splicing is absent in untreated (e.g., mock-transfected or transfected with a scrambled ASO) patient fibroblasts. Quantifying these results shows that AT008 had robust activity with recovery of wild-type splicing (i.e., up to 50% of total transcripts), with AT009 and AT007 generating the next highest percentages of wild-type slicing (approximately 40% and 32% of total transcripts or about 0.8 and 0.65 the amount observed for AT008, respectively) (FIG. 10B).

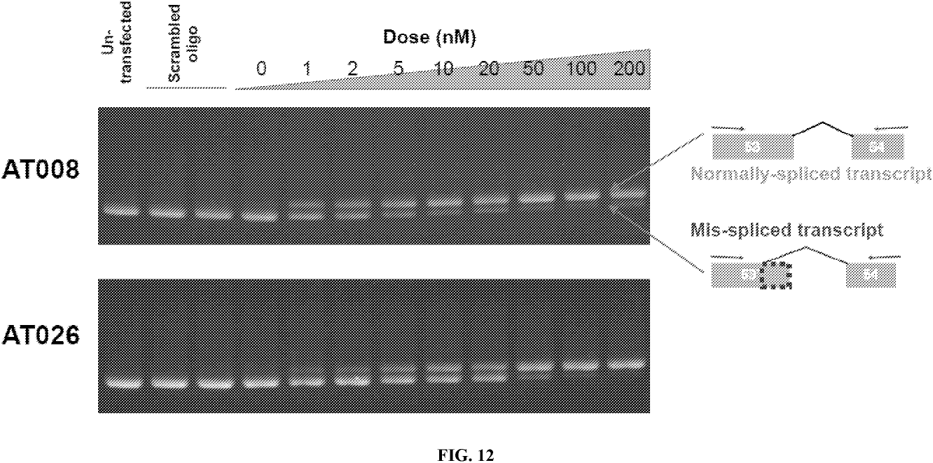

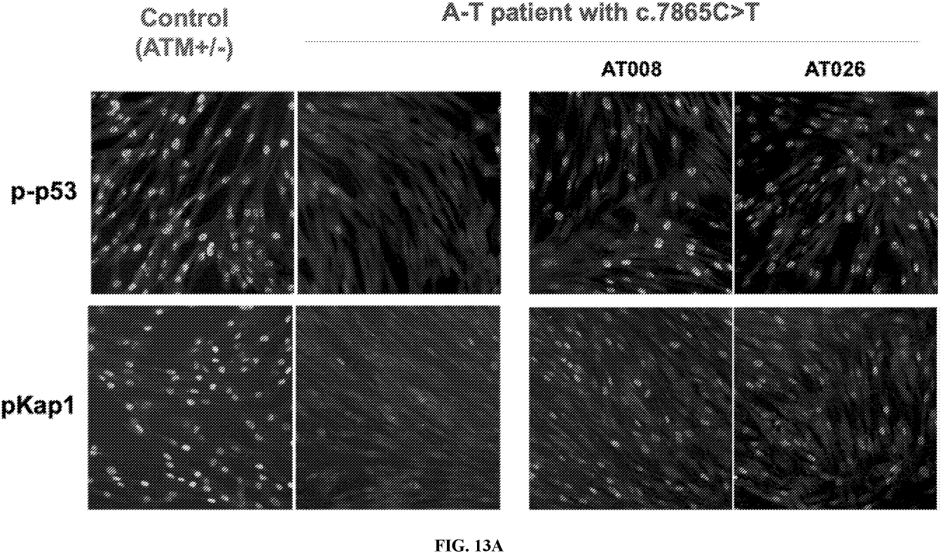

After identifying AT008 as the lead candidate antisense oligonucleotide, additional oligonucleotides (AT022-AT041) were synthesized and tested to assess efficacy (see Table 2). Several of the candidate optimized oligonucleotides showed similar efficacy to AT008 (FIG. 11). To determine if the antisense oligonucleotide-mediated recovery of wild-type splicing was dose dependent, AT008 and AT026 were transfected into cultured patient fibroblasts at doses ranging between 0 nM and 200 nM. Referring to FIG. 12, RT-PCR results show that normally-spliced exon 53 products are observed at doses as low as 1 nM for both antisense oligonucleotides. At higher doses of either antisense oligonucleotide, wild-type ATM transcripts are the predominant splice product.

### Example 5: Functional Screening of AT008 and AT026

To test whether delivery of antisense oligonucleotides would have a functional impact on cells carrying the NM_000051.3(ATM):c0.7865C>T mutation, AT008 and AT026 were evaluated using the functional assay described in Example 3 (see also FIG. 6). Referring to FIG. 13A, fibroblast cells from the patient’s heterozygous parent and fibroblasts from the patient treated with either AT008 or AT026 exhibited phosphorylated Kap1 and p53 after the cells were subjected to irradiation (1.5 Gy). The staining pattern demonstrates recovered activation and nuclear localization of phosphorylated p53 and phosphorylated Kap1 following treatment with AT008 and AT026. The assay was repeated using fibroblast cells derived from control fibroblasts derived from non-patient (carrier of a ATM mutation), A-T patient fibroblasts without NM_000051.3(ATM):c0.7865C>T, and A-T patient fibroblasts with NM_000051.3(ATM):c0.7865C>T (heterozygous). As expected, the cultured fibroblasts heterozygous mother displayed a strong signal for Kap1 and p53 phosphorylation after exposure to radiation, while untreated fibroblasts from the proband having the NM_000051.3(ATM):c0.7865C>T mutation and the proband from Examples 1-3 had weak signals for both phosphorylated Kap1 and p53 (FIG. 13B). Transfection of fibroblasts having the NM_000051.3(ATM):c0.7865C>T mutation with AT007, AT008, AT022, and AT026 all exhibited increased signal for phosphorylated Kap1 and p53 compared to the untreated controls. Quantifying the results of FIG. 13B shows that pKap1 activation in fibroblasts derived from the proband is improved by delivery of the antisense oligonucleotides relative to untreated controls but is less than 40% of the activation seen in the fibroblasts derived from the control (non-A-T patient; ATM mutation carrier) (FIG. 13C). p53 activation in fibroblasts derived from the proband was approximately 80% of the p53 activation observed in the fibroblasts derived from the proband’s mother for all oligonucleotide tested (FIG. 13D).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An antisense nucleobase oligomer comprising 8-40 nucleobases, wherein at least 90% of said nucleobases or more than 8 consecutive nucleobases of the oligomer are complementary to a nucleic acid sequence in an Ataxia-Telangiectasia Mutated (ATM) allele comprising a mutation associated with aberrant splicing.

2. The antisense nucleobase oligomer of claim 1, wherein the oligomer comprises a modified linkage selected from the group consisting of phosphorothioate, methylphosphonate, phosphodiester, phosphotriester, and phosphorodithioate linkages.

3. The antisense nucleobase oligomer of claim 2, wherein the modified linkage is a phosphorothioate linkage.

4. The antisense nucleobase oligomer of claim 3, wherein the antisense nucleobase oligomer is a mixture of one or more stereopure oligomers with a defined sequence.

5. The antisense nucleobase oligomer of of claim 1, wherein the antisense nucleobase oligomer comprises a modified nucleobase.

6. The antisense nucleobase oligomer of claim 5, wherein the modified nucleobase is 5-methyl cytosine.

7. The antisense nucleobase oligomer of claim 1, further comprising at least one modified sugar moiety.

8. The antisense nucleobase oligomer of claim 7, wherein the modified sugar moiety is a 2′-O-methoxyethyl group, a 2′-O-methyl, 2′-dimethylaminooxyethoxy, 2′-dimethylaminoethoxyethoxy, 2′-fluoro or 2′-acetamide modification group.

9. The antisense nucleobase oligomer of of claim 1, wherein the oligomer comprises locked nucleic acids.

10. The antisense nucleobase oligomer of of claim 1, wherein the oligomer is a morpholino, thiomorpholino, or peptide nucleic acid.

11. The antisense nucleobase oligomer of claim 1, wherein the mutation is a NM_000051.3(ATM):c0.5762ins137 mutation or a NM_000051.3(ATM):c0.7865C>T mutation.

12-14. (canceled)

15. The antisense nucleobase oligomer of claim 1, wherein the antisense nucleobase oligomer comprises at least 10 nucleobases of the following sequences or consists of the following sequences: TTCTTCAGGATCTTATTCAGCA SEQ ID NO: 1); ATTCTTCAGGATCTTATTCAGC (SEQ ID NO: 2); TCTTCAGGATCTTATTCAGCA (SEQ ID NO: 3); TTCTTCAGGATCTTATTCAGC (SEQ ID NO: 4); CTTCAGGATCTTATTCAGCA (SEQ ID NO: 5); TTCTTCAGGATCTTATTCAG (SEQ ID NO: 6); CTTCAGGATCTTATTCAGC (SEQ ID NO: 7); TCTTCAGGATCTTATTCAG (SEQ ID NO: 8); TTCAGGATCTTATTCAGC (SEQ ID NO: 9); CTTCAGGATCTTATTCAG (SEQ ID NO: 10); TCTTCAGGATCTTATTCA (SEQ ID NO: 11); TCAGGATCTTATTCAGC (SEQ ID NO: 12); and TCTTCAGGATCTTATTC (SEQ ID NO: 13).

16. (canceled)

17. The antisense nucleobase oligomer of claim 1, wherein the antisense nucleobase oligomer comprises at least 10 nucleobases of the following sequences or consists of the following sequences: AATATAAGCATCACAAAGTACC (SEQ ID NO: 15); ATAAGCATCACAAAGTACCTC (SEQ ID NO: 16); TATAAGCATCACAAAGTACCT (SEQ ID NO: 17); ATATAAGCATCACAAAGTACC (SEQ ID NO: 18); AATATAAGCATCACAAAGTAC (SEQ ID NO: 19); TAAGCATCACAAAGTACCTC (SEQ ID NO: 20); ATAAGCATCACAAAGTACCT (SEQ ID NO: 21); TATAAGCATCACAAAGTACC (SEQ ID NO: 22); ATATAAGCATCACAAAGTAC (SEQ ID NO: 23); AAGCATCACAAAGTACCTC (SEQ ID NO: 24); TAAGCATCACAAAGTACCT (SEQ ID NO: 25); ATAAGCATCACAAAGTACC (SEQ ID NO: 26); TATAAGCATCACAAAGTAC (SEQ ID NO: 27); ATATAAGCATCACAAAGTA (SEQ ID NO: 28); AGCATCACAAAGTACCTC (SEQ ID NO: 29); AAGCATCACAAAGTACCT (SEQ ID NO: 30); TAAGCATCACAAAGTACC (SEQ ID NO: 31); ATAAGCATCACAAAGTAC (SEQ ID NO: 32); TATAAGCATCACAAAGTA (SEQ ID NO: 33); and AAGCATCACAAAGTACC (SEQ ID NO: 34).

18. (canceled)

19. A set of antisense nucleobase oligomers comprising 2 or more of the antisense nucleobase oligomers of claim 1.

20. A pharmaceutical composition comprising an effective amount of the antisense nucleobase oligomers of claim 1 and a pharmaceutically acceptable excipient.

21. A method of restoring wild-type splicing of an ATM allele comprising a mutation associated with ataxia telangiectasia in a cell, the method comprising:
contacting the cell with an effective amount of the antisense nucleobase oligomer of claim 1, thereby restoring wild-type splicing.

22. (canceled)

23. The method of claim 21, wherein the mutation is in a splice acceptor site, a splice donor site, exonic splicing enhancer (ESE), or a splicing regulatory element.

24. (canceled)

25. The method of claim 21, wherein the mutation is a NM_000051.3(ATM):c0.5762ins137 or NM_000051.3(ATM):c0.7865C>T mutation.

26. (canceled)

27. A method of treating ataxia telangiectasia in a subject, the method comprising:
administering to the subject an effective amount of an antisense nucleobase oligomer of claim 1.

28. The method of claim 27, wherein the subject comprises a mutation associated with ataxia telangiectasia that is a NM_000051.3(ATM):c0.5762ins137 mutation or NM_000051.3(ATM):c0.7865C>T mutation.

29-31. (canceled)

32. An isolated cell comprising an antisense oligomer of claim 1 and a mutation associated with ataxia telangiectasia.

33-36. (canceled)
